(19) United States
(12) Patent Application Publication
(10) Pub. No.: US 2007/0072255 A1
(43) Pub. Date: Mar. 29, 2007

(54) Toxicity detector for liquid consumable by humans

(76) Inventors: Prasad S. Kodukula, Chicago, IL (US); Charles R. Stack, Aurora, IL (US)

Correspondence Address: John C. McMahon, PO Box 30069, Kansas City, MO, 64112, US

(21) Appl. No.: 11/432,605

(22) Filed: May 11, 2006

Related U.S. Application Data
(62) Division of application No. 10/317,363, filed on Dec. 12, 2002.

(51) Int. Cl. C12Q 1/26; C12Q 1/04; G06F 19/00

(52) U.S. Cl. 435/25; 435/34; 702/19

(57) ABSTRACT

A process provides for testing for certain toxins and poisons in liquids, gases and solids that are consumed by humans or otherwise come in contact with humans. In the process, the material to be tested is mixed with aerobic microorganisms in a biomass to form a mixture. The mixture is observed to determine if a preselected change in the biomass occurs. Preferably, the mixture is observed to determine if the biomass has a normal rate of respiration.

### CROSS REFERENCE TO RELATED APPLICATIONS

The present application is a divisional application of U.S. application for patent Ser. No. 10/317,363, filed Dec. 13, 2002, now U.S. Pat. No. ______.

### BACKGROUND OF THE INVENTION

The present invention is directed to a method of determining if a liquid used by humans contains toxins by adding an aerobic microorganism that is effected by such toxins to the liquid and then determining if there is a change in the microorganism, especially in the respiration rate thereof. The liquid may be potable water, other human consumable beverages, and solids or gases that are contained in a carrier liquid.

Because of terrorist activities, there is a general widespread concern that various beverages or edible products may in some way be tampered with and poisoned prior to delivery to the public. For example, various chemicals may be placed in a fresh or potable water supply prior to or even after conventional treatment at a facility preparing the water for delivery to the public. Other beverages such as milk may be tampered with at various points along the production path such as at a farm, during transport, during pasteurization, in storage and at or after bottling. Beer, carbonated beverages, fruit juices and even bottled water can likewise be tampered with so as to include a poison of some type. Certain foods may also be poisoned. Still further, gases such as air circulated in a building, subway or otherwise put in contact with people via food or drink, such as carbon dioxide used to carbonate soft drinks, may have some type of poison added to them.

Even when deliberate steps are not taken by a person to contaminate or poison beverages and foodstuff, it is very possible for it to happen accidently. This is especially true due to the complexity of processes and the widespread use of various materials that can kill or injure anyone who ingests or otherwise comes in contact with the materials. Various governmental regulations attempt to isolate consumables from potential hazardous materials, but the maxim that is embodied in Murphy's law is true in that events that are very unlikely to happen will in fact at times occur in such a way to make the unlikely or even seemingly impossible happen.

Therefore, it is desirable to be able to test various liquid or gas streams and even solids that can be broken down and carried in a liquid to ensure that certain poisons or toxins are not contained therein.

### SUMMARY OF THE INVENTION

The present invention is directed to a process for detecting toxic substances that are inherently present in or deliberately or accidentally introduced into any liquid stream such as waters, beverages, milk, etc. The process can also detect such toxic substances in gases and solid materials after they have been dissolved or slurried into a liquid carrier. The process for detecting such toxins may be either a batch mode or a continuous mode in nature. Continuous mode operation allows on-line, real-time detection of toxins in conjunction with warning alarms sent to pre-identified receivers via land-line, wireless and satellite-based communication devices. Either mode of operation is capable of identifying a tainted liquid stream and selectively allowing the stoppage of usage, directing the liquid to a storage tank, adding chemicals to negate the substance or toxins tainting the stream and/or continuing normal operation if the data is within acceptable pre-determined parameters.

In the process, a mixing tank receives a test liquid (or carrier liquid with a gas, air or solid material therein) of interest that may contain toxic substances. A biomass containing microorganism is added to the liquid along with nutrients where needed to feed the biomass outside the liquid. A sources of oxygen is added to the liquid, such as air. Thereafter, the microorganisms are observed or an activity of the microorganism is observed to see if there is a decrease in activity from an expected norm or bench mark. For example, respiration of the microorganisms is observed to see if the respiration decreases.

An apparatus of the type used herein for the process of the invention is disclosed in U.S. Pat. No. 5,106,511 which is incorporated herein by reference. While the noted patent discloses an apparatus that is effective in accordance with the invention, it is foreseen the other device may be utilized in accordance with the invention.

### OBJECTS AND ADVANTAGES OF THE INVENTION

Therefore, the objects of the present invention are: to provide a detection process for detecting a poison or toxin in a liquid that is consumable by a human or animal or which comes in contact with humans or animals, including fresh water supplies, beverages, liquid carriers into which gas is absorbed for testing and liquid carriers into which solids are dissolved or slurried; to provide such a detection process that can be used to simultaneously test for a wide range of such poisons or toxins; to provide such a detection process that sends out alarms to persons in response to the detection of poisons or toxins; to provide such a detection process wherein alarms are sent on a hierarchal basis depending on severity of any projected problem to different persons or to persons who can best handle a particular problem with recommendations on how to avoid or reduce the severity of the problem; to provide such a detection process that tests for preselected changes in microorganisms that are added to the liquid, especially inhibition or decrease in oxygen consumption of the microorganism, in order to indicate that the microorganisms are encountering a toxin or poison; to provide a method for the detection of toxic substances inherently present in or introduced deliberately or accidentally into any liquid stream such as waters, beverages, milk, etc; and to provide such a method which is relatively easy to use and especially suited for the intended purpose thereof.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is a schematic drawing of a process for testing fresh water in accordance with the present invention.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

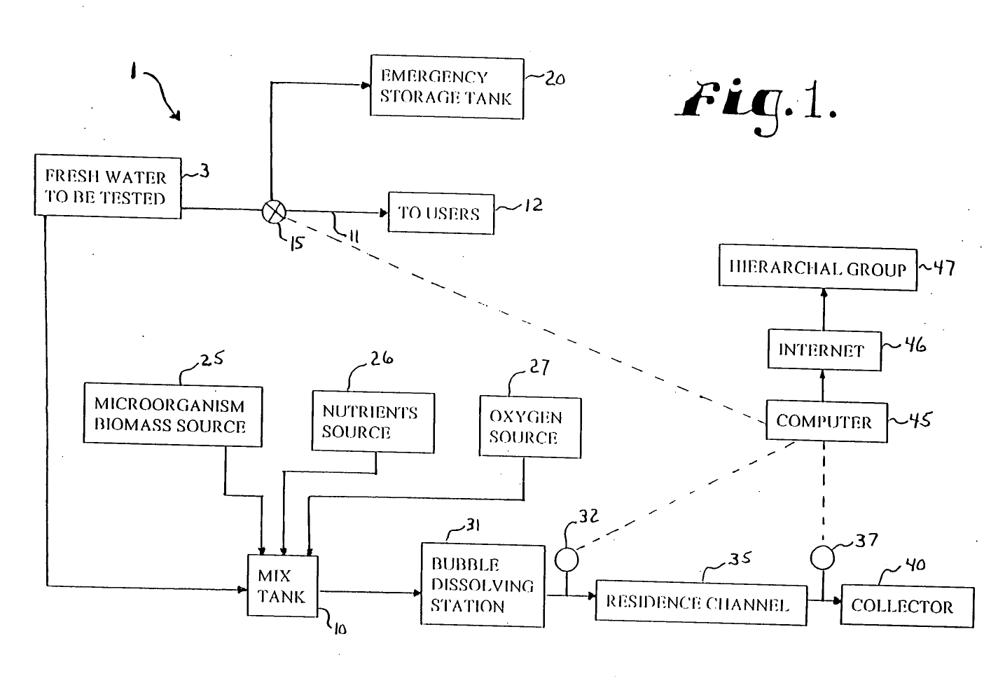

Illustrated in FIG. 1 is a schematic of a process for the detection of toxicity or inhibition in a liquid in accordance with the present invention and generally represented by the reference numeral 1. A liquid, in the present embodiment a fresh water supply source 3 is tested by the process.

In this case the source 3, is water leaving a water treatment facility; however, it is foreseen that testing could be as the water enters the treatment facility or as the water enters a large end user such as a hotel. A slip stream 4 of the water is directed to a mix tank 10. Under normal operating conditions, the water from source 3 will flow in a main line 11 to water users, identified by the box 12. Normally the end user will be a living human. The main water line 11 also has a diverter valve 15 that can be activated either by operator or automatically by the process 1 to divert the water from the main line 11 to a storage pond or tank 20 should the water be found to have a toxin or poison by the process of the invention.

The mix tank 10 also receives a microorganism biomass from a microorganism source 25, nutrients from a nutrients source 26 and oxygen from an oxygen source 27 for respiration of the microorganisms. The biomass source 25 may be any readily available biomass, such as material directly from a waste water digester, freeze dried biomass, powdered biomass, separate cultures, or the like. The nutrients source will normally function as a food source for the microorganisms in the biomass and such are commonly known. The types and amounts of specific nutrients that must be added to liquid to be tested depends heavily on the content of the liquid. Even when the liquid is potable water, certain nutrients such as minerals may already exist in the liquid. A main component of such nutrients that is required and will have to be added any to potable water to be tested is a source of organic material or carbon source for the microorganisms. Carbon may be derived form many different organic sources, including fermentable sugars (especially glucose), simple sugars (such as sucrose, fructose and the like), organic acids (such as acetic acid or lactic acid), alcohols (such as ethanol) and many short chained organic compounds. Often, it is desirable for the nutrients to include a source of nitrogen and a source of phosphorus. Preferably, these nutrients are present in a ratio by weight of one hundred parts carbon to five parts nitrogen to one part phosphorus. It is also foreseen that the nutrient source may also include trace nutrients such as iron, magnesium, potassium, calcium, sulfur and the like. The source for the nitrogen may include diammonium phosphate, nitric acid, ammonium chloride, sodium nitrate, ammonium hydroxide and other similar sources. The phosphorus source may be phosphoric acid, trisodium phosphate, diammonium phosphate and the like. Still further, the nutrients may include growth factors and amino acids, pyrimidines, vitamins and the like. A good source of many of the additives is yeast extract. In some situations the pH of the liquid to be tested must be raised or lowered and a source of a base or acid is added which may be a dual use component, such as ammonium hydroxide (to supply both a base and nitrogen) or phosphoric acid (to supply both an acid and phosphorus. It is foreseen also that the process may not require the addition of a nutrients source in some situations or that certain test liquids may not require nutrients. For example, some liquids such as milk may provide sufficient nutrients for the microorganisms without the need to add additional nutrients.

Oxygen is added from the oxygen source 27 in sufficient amount to ensure the dissolved oxygen content of the liquid in the mix tank 10 is sufficient to supply the microorganisms in the biomass with oxygen during the process. In some instances the liquid may have sufficient oxygen and not require more.

Biomass is added from the biomass source 25. In the present embodiment the biomass is a cultured bacteria that is added in a fixed amount to the water. However, it is foreseen that the biomass could be from any other suitable source for the type discussed further below.

In the mix tank 10, a mixture of the fresh water and microorganisms is thus produced in some instances with added oxygen and nutrients. It is also foreseen that in some instances a buffer will be added to the mix tank to reduce pH variance. The mixture flows into a bubble dissolving station 31 so as to allow gas bubbles in said mixture to fully dissolve into the mixture. Thereafter, the mixture encounters a first sensor 32 that measures the dissolved oxygen within the mixture. The mixture then flows into a reactor or residence channel 35. In the illustrated usage, it is desirable to allow the mixture to remain within the channel 35 for a length of time to allow for sufficient aerobic reaction of the mixture for example, 30 minutes. Having sufficiently reacted and upon exiting the channel 35, a second sensor 37 measures the dissolved oxygen of the mixture and the test mixture exits to sample collector 40 or the sample is discharged to the sewer. Both the first and second sensors 32 and 37 are connected to a computer 45 that receives the dissolved oxygen data to calculate an oxygen consumption rate which is then compared to a pre-determined figure or a baseline. If the resulting calculation is sufficiently outside expected parameters, the computer 45 sends out an alarm signal. The alarm may control the valve 15 so as to divert flow of water from the users 12 to the storage tank 20 or may warn an operator who manually changes the flow. Furthermore, the computer 45 is connected via the internet 46 or other communication means to a hierarchal group 47 of pre-identified receivers. The computer 45, depending on its programming, may send alarm signals in real-time to this group of 47 receivers to notify selected personnel of a potential toxicity associated with the liquid.

It is foreseen that the majority of substances to be tested for toxicity will be deficient or completely free of any inherent microorganisms, so the device will require ongoing “seeding” of organisms that are vulnerable to suspected toxins. Typical microorganisms will include different forms of bacteria, protozoans, algae, and other single-cell life forms from yeast, mold, plant or animal sources. Certain forms of bacteria may include bacillus, pseudomonas, and others. Certain forms of protozoans may include paramecium, aspidiscus, and others. Certain forms of algae may include chlorella, volvox, and others. Still further, it is noted that the microorganisms may be single-cell or multi-cell organisms and that there may even be mammal cells or tissue that would be more susceptible to toxins that may harm humans.

It is also foreseen that the method of the present invention can be used to test for toxins or poisons in liquids and other materials including: surface and groundwater supplies such as water from lakes, rivers, reservoirs, etc; potable water from public water sources, distribution systems, wells, etc; beverages including milk, soft drinks, fruit juices, alcoholic products etc; pharmaceuticals and medical products including drugs, IV solutions, etc; gases dissolved in a liquid carrier such as air including the atmosphere of industrial, residential, health care, military, government and other public locations susceptible to poisoning; other gases such as pure gases including oxygen supplies for hospitals, carbon dioxide for beverage carbonation, nitrogen for modified atmospheric packaging and additional gases that might impact the public health if contaminated; solids dissolved in a liquid or suspended in a slurry in a carrier liquid such as water, including food ingredients such as sugar, flour, whey powder, spices, preservatives, etc; other solids including soil, agricultural products (grain, seed, etc), meats, baked goods, cloth and clothing, etc which in some instances must first be powdered or broken into minute particles.

It is to be understood that while certain forms of the present invention have been illustrated and described herein, it is not to be limited to the specific forms or arrangement of parts described and shown.

What is claimed and desired to be secured by Letters Patent is as follows:

1. A process for the detection of toxic substances within a first liquid that come in contact with an animal; said process comprising the steps of:
a) providing a channel including at least first and second spaced sensors for operably measuring the fluid's dissolved oxygen with said first sensor being located at an entrance of said channel;
b) providing a second liquid having a microorganism biomass therein;
c) mixing said first and second liquids so as to form a mixture;
d) subsequent to step c, placing said mixture in said channel and measuring an initial dissolved oxygen content of said mixture with said first sensor;
e) flowing said mixture through said channel;
f) analyzing a final dissolved oxygen within said mixture at an exit of said channel with said second sensor; and
g) comparing the initial dissolved oxygen content at said first sensor to the final dissolved oxygen content at said second sensor to determine a reduction in dissolved oxygen content between said first and second sensors.

2. The process according to claim 1 including the step of:
a) comparing said reduction in dissolved oxygen content to a baseline reduction in oxygen content to determine if the actual reduction in dissolved oxygen content is outside of a preselected baseline.

3. The process according to claim 2 including the step of:
a) sending an alarm signal in response to said reduction in dissolved oxygen content being greater than said baseline.

4. The process according to claim 1 including the step of:
a) including nutrients in said second liquid for said microorganism biomass.

5. The process according to claim 1 including the step of:
a) providing an oxygen source for injecting oxygen into said mixture prior to said mixture entering said channel.

6. The process according to claim 1 including the step of:
a) providing a holding region between mixing and said first sensor though which said mixture must flow so as to allow gas bubbles in said mixture to fully dissolve into said mixture prior to flowing past said channel first sensor.

7. The process according to claim 1 including the step of:
a) batching said process.

8. The process according to claim 1 including the step of:
a) operating said process as a continuous process.

9. The process according to claim 8 including the steps of:
a) providing on-line, real-time sensor readings by comparing readings of said first and second sensor with a preselected baseline to provide for detection of toxic substances within said first liquid due to a significant reduction in respiration.

10. The process according to claim 9 including the step of:
a) sending a warning signal if the difference in said senor readings is less than said baseline.

11. The process according to claim 1 including the step of:
a) providing a computer that is adapted to communicate with a recorder through which information produced by said first and second sensors is recorded.

12. The process according to claim 11 including the step of:
a) utilizing said computer to react to information submitted by said first and second sensors to trigger an alarm system if the respiration rate is less than a preselected baseline rate.

13. The process according to claim 12 including the step of:
a) diverting flow of a stream of said first liquid found to possibly include a toxic substance to an emergency storage tank for further investigation.

14. The process according to claim 12 including the step of:
a) using said computer to communicate with pre-identified receivers at selected land-line, wireless and satellite-based communication devices to provide information regarding analysis of the first liquid to remote locations.

15. The process according to claim 14 including the step of:
a) communicating with said pre-identified receivers on the basis of hierarchal groupings whereby said computer communicates with the receivers based on the severity of toxicity based on the disparity between the baseline and the actual respiration rate.

16. The process according to claim 1 including the step of:
a) selecting potable water as the first liquid.

17. The process according to claim 1 including the step of:
a) initially forming said first liquid by mixing a carrier liquid with a potentially toxic solid to form a slury.

18. The process according to claim 1 including the step of:
a) initially forming said first liquid by mixing a potentially toxic gas with a carrier liquid such that said potentially toxic gas dissolves in said carrier liquid.

19. A process for testing potable water supplies comprising the steps of:
a) providing a test quantity of potable water;
b) adding said quantity of potable water to a bacterial biomass to form a mixture;
c) adding a nutrient for said biomass to said mixture;
d) adding a source of oxygen to said mixture;
e) thereafter allowing mixture to react for a preselected period of time; and
f) measuring usage of oxygen within said biomas during said preselected period of time.

20. The process according to claim 19 including the step of:
a) providing said bacterial mass from a bacterial culture.

21. The process according to claim 29 including the step of:
a) providing said bacterial mass from living sludge taken from a wastewater-treatment plant.

22. A method of detecting toxic substances in potable water comprising the steps of:
a) adding a microorganism biomass from a biological reactor into said water to form a mixture;
b) adding a nutrient for the biomass to said mixture;
c) adding a source of oxygen to said mixture;
d) thereafter routing said mixture to a holding tank for sufficient time to dissolve gas bubbles that may exist in the mixture;
e) thereafter transferring said mixture to a channel;
f) measuring the dissolved oxygen content of the mixture near an entry to said channel with a first sensor;
g) measuring the dissolved oxygen content of the mixture near an exit of said channel with a second sensor;
h) transmitting data from the first and second sensors to a computer where oxygen respiration in said channel is computed;
i) comparing the computed respiration to a preselected baseline;
j) providing an alarm signal if the computed respiration is significantly below said preselected baseline.

23. In a process for testing a liquid that is initially generally free of microorganisms for certain toxins, the improvement comprising the steps of:
a) adding an aerobic microorganism biomass that is susceptible to said certain toxins to said liquid; and thereafter
b) testing the respiration rate of said microorganism and comparing the respiration rate to a baseline.
